(19) United States
(12) Patent Application Publication
(10) Pub. No.: US 2024/0130770 A1
(43) Pub. Date: Apr. 25, 2024

(54) MORPHABLE BONE FIXATION DEVICE, SYSTEM AND METHOD

(71) Applicant: Board of Regents, The University of Texas System, Austin, TX (US)

(72) Inventors: Farshid Alambeigi, Austin, TX (US); Amir Hossein Eskandari Shahrabi, Montreal (CA)

(21) Appl. No.: 18/569,842

(22) PCT Filed: Jun. 14, 2022
(86) PCT No.: PCT/US22/33552

§ 371 Date: Dec. 13, 2023

(51) Int. Cl. A61B 17/86; A61B 17/00

(52) U.S. Cl. CPC A61B 17/8635; A61B 17/00234; A61B 17/864; A61B 17/866; A61B 2017/003

(57) ABSTRACT

A bone fixation device comprises a continuum morphable hollow shell structure including a proximal end and a distal end, an implantation head at the proximal end and a screw thread positioned external to the shell structure between the implantation head and a screw tip positioned at the distal end. A bone fixation system comprises a bone fixation device as described above, at least one reinforcement wire configured for insertion into the bone fixation device, and a castable filler material configured for insertion into the bone fixation device. A bone fixation method comprises fabricating a bone fixation device as described above, drilling the implantation trajectory, implanting the bone fixation device, implanting at least one reinforcement wire within the bone fixation device, and injecting the bone fixation device with a castable filler material via an aperture of the bone fixation device.

### CROSS-REFERENCE TO RELATED APPLICATIONS

This application claims priority to U.S. provisional application No. 63/211,015 filed on Jun. 16, 2021, incorporated herein by reference in its entirety.

### BACKGROUND

Screw implants are commonly used to stabilize bone fractures, reconstruct bone after tumor resection or destruction from infection, and treat congenital and acquired degenerative diseases. Screw fixation usually inserts rigid bone screws through strong cortical bone and into the more porous cancellous bone. The screws can then be rigidly connected with locking rods to ideally provide a stable fixation and load sharing feature before a robust bone fusion or healing occurs. However, screw fixation suffers from various types of complications and failures, including but not limited to screw misplacement, screw fracture, bone fracture, and loosening and pullout of screw implants. In particular, while loosening and pullout of screw implants is a prevalent problem in osteoporotic bone, it is also a common occurrence in bones with normal and healthy bone mineral density (BMD).

The reasons for the inadequacy of screw implants in bone are manifold. Screw implant sites in bone must deal with narrow and confined anatomical constraints, limiting the angles of approach for the screws. Nerves and blood vessels also must be avoided from the screw path. Additional obstacles are regions of low BMD. Fixation strength and quality of screw implant fixation directly depend on the BMD of an implant site. Traditional drilling instruments and screws are rigid and lack the sufficient dexterity to navigate the aforementioned anatomical constraints, limiting implant trajectories to linear paths that often lead to screw misplacement and nerve injury and necessarily cross low BMD regions.

Thus, there is a need in the art for improved devices and methods for implant fixation in bone that are adapted for a subject's bone mineral density. The present invention meets this need.

### SUMMARY

Some embodiments of the invention disclosed herein are set forth below, and any combination of these embodiments (or portions thereof) may be made to define another embodiment.

In one aspect, a bone fixation device comprises a continuum morphable hollow shell structure including a proximal end and a distal end, an implantation head at the proximal end including an aperture, and a screw thread positioned external to the shell structure between the implantation head and a screw tip positioned at the distal end.

In one embodiment, the implantation head comprises a screw head. In one embodiment, the shell structure is cylindrical. In one embodiment, the shell structure has an outer diameter in the range of 2 mm to 20 mm, an inner diameter in the range of 1.5 mm to 19.5 mm, and a wall thickness in the range of 0.1 mm to 2 mm. In one embodiment, the screw thread has a thread pitch in the range of 1 mm to 2 cm. In one embodiment, the shell structure has a length in the range of 1 cm to 50 cm. In one embodiment, the shell structure comprises at least one of ABS, PLA, FormLabs inc. biomaterial (BioMed Clear Resin, BioMed Amber Resin), StrataSys biomaterial (Biocompatible MED625FLX, Biocompatible MED610, Biocompatible VeroGlaze MED620), polyamide, PEEK, titanium, nitinol, and cobalt-chrome alloys. In one embodiment, the shell structure is a 3D printed part.

In another aspect, a bone fixation system comprises a bone fixation device as described above, at least one reinforcement wire configured for insertion into the bone fixation device, and a castable filler material configured for insertion into the bone fixation device.

In one embodiment, the system further comprises at least one strain gauge configured for insertion into the bone fixation device. In one embodiment, the filler material comprises a low melting point alloy. In one embodiment, the filler material comprises at least one of Field's metal, dental amalgam, and resin-based composite. In one embodiment, the at least one reinforcement wire comprises at least one of Nitinol, stainless steel, titanium, and carbon fiber. In one embodiment, the stiffness of the system is configurable based on a finite element analysis modeled developed based on a target bone mineral density and target bone anatomy by changing the material properties of the castable filler material.

In another aspect, a bone fixation method comprises fabricating a bone fixation device as described above, drilling an implantation trajectory, implanting the bone fixation device, implanting at least one reinforcement wire within the bone fixation device, and injecting the bone fixation device with a castable filler material via an aperture of the bone fixation device.

In one embodiment, the method characterization of the target bone tissue further comprises characterizing a target bone tissue including identifying regions of osteoporotic bone and bone with low mineral density, and forming the implantation trajectory based on the characterization. In one embodiment, the implantation trajectory is configured to avoid the identified regions of osteoporotic bone and bone with low mineral density. In one embodiment, the step of characterizing the target bone tissue comprises the steps of performing one or more quantitative computed tomography (QCT) scans on the target bone tissue, converting the one or more QCT scans into a three-dimensional finite element model of the target bone tissue, and demarcating osteoporotic regions or low bone mineral density regions in the three-dimensional finite element model. In one embodiment, the bone fixation device is fabricated by at least one of additive manufacturing and molding. In one embodiment, the filler material comprises a low melting point alloy. In one embodiment, the filler material comprises at least one of Field's metal, dental amalgam, and resin-based composite. In one embodiment, the injecting of the filler material into the bone fixation device is performed in vivo. In one embodiment, the at least one reinforcement wire comprises at least one of Nitinol, stainless steel, titanium, and carbon fiber. In one embodiment, the drilling is performed with a flexible steerable drilling robot configured to create at least one of a straight, a curved, or a complex trajectory.

### BRIEF DESCRIPTION OF THE DRAWINGS

The following detailed description of exemplary embodiments of the invention will be better understood when read in conjunction with the appended drawings. It should be understood, however, that the invention is not limited to the precise arrangements and instrumentalities of the embodiments shown in the drawings.

FIG. 1 depicts an exemplary bone fixation system in accordance with some embodiments.

FIG. 2A depicts an exemplary bone fixation device of an exemplary bone fixation system in accordance with some embodiments.

FIG. 2B depicts schematics of exemplary engagement members in accordance with some embodiments.

FIG. 3 depicts an exemplary process for implantation of an exemplary bone fixation system in accordance with some embodiments.

FIG. 4 depicts an exemplary bone fixation method utilizing an exemplary bone fixation system in accordance with some embodiments.

FIG. 5 depicts experimental results of an exemplary bone fixation system configured in a J-shape trajectory implanted in a model vertebra in accordance with some embodiments.

FIG. 6 depicts experimental results of an exemplary bone fixation system configured in a U-shaped trajectory implanted in a model vertebra in accordance with some embodiments.

FIG. 7 depicts experimental results of an exemplary bone fixation system configured as a long and curved trajectory implanted in a model upper pelvis in accordance with some embodiments.

FIG. 8 depicts experimental results of an exemplary bone fixation system configured as curved and long trajectory implanted in a model right pelvis in accordance with some embodiments.

### DETAILED DESCRIPTION

The present invention provides bone fixation devices, systems and methods that can be implanted into the curved trajectories to enhance implant fixation in bone. The curved implantation trajectories can avoid regions of low bone mineral density, such that implanted bone fixation system driven into the curved implantation trajectories are anchored in regions of high bone mineral density to improve the stability of bone fixation. The bone fixation system is suitable for several applications, including but not limited to spinal fixation, orthopedic bone fixation, and neurosurgery.

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

The terms “proximal,” “distal,” “anterior,” “posterior,” “medial,” “lateral,” “superior,” and “inferior” are defined by their standard usage indicating a directional term of reference. For example, “proximal” refers to an upper location from a point of reference, while “distal” refers to a lower location from a point of reference. In another example, “anterior” refers to the front of a body or structure, while “posterior” refers to the rear of a body or structure. In another example, “medial” refers to the direction towards the midline of a body or structure, and “lateral” refers to the direction away from the midline of a body or structure. In some examples, “lateral” or “laterally” may refer to any sideways direction. In another example, “superior” refers to the top of a body or structure, while “inferior” refers to the bottom of a body or structure. It should be understood, however, that the directional term of reference may be interpreted within the context of a specific body or structure, such that a directional term referring to a location in the context of the reference body or structure may remain consistent as the orientation of the body or structure changes.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

Some aspects of the present invention may be made using an additive manufacturing (AM) process. Among the most common forms of additive manufacturing are the various techniques that fall under the umbrella of “3D Printing”, including but not limited to stereolithography (SLA), digital light processing (DLP), fused deposition modelling (FDM), selective laser sintering (SLS), selective laser melting (SLM), electronic beam melting (EBM), and laminated object manufacturing (LOM). These methods variously “build” a three-dimensional physical model of a part, one layer at a time, providing significant efficiencies in rapid prototyping and small-batch manufacturing. AM also makes possible the manufacture of parts with features that conventional subtractive manufacturing techniques (for example CNC milling) are unable to create.

Suitable materials for use in AM processes include, but are not limited to, using materials including but not limited to nylon, polyethylene terephthalate (PET), acrylonitrile butadiene styrene (ABS), resin, polylactic acid (PLA), polystyrene, and the like. In some embodiments, an AM process may comprise building a three-dimensional physical model from a single material, while in other embodiments, a single AM process may be configured to build the three-dimensional physical model from more than one material at the same time.

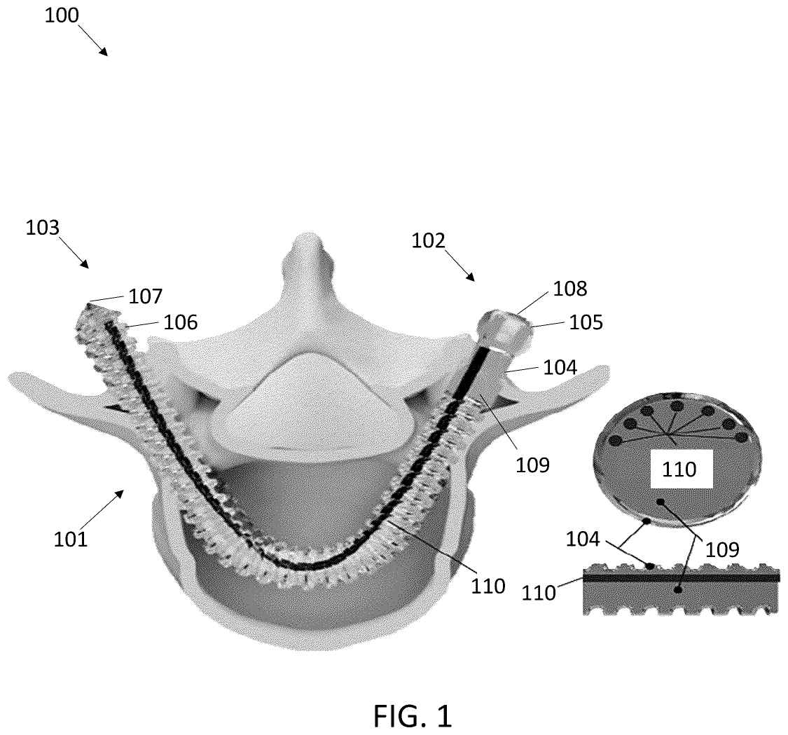

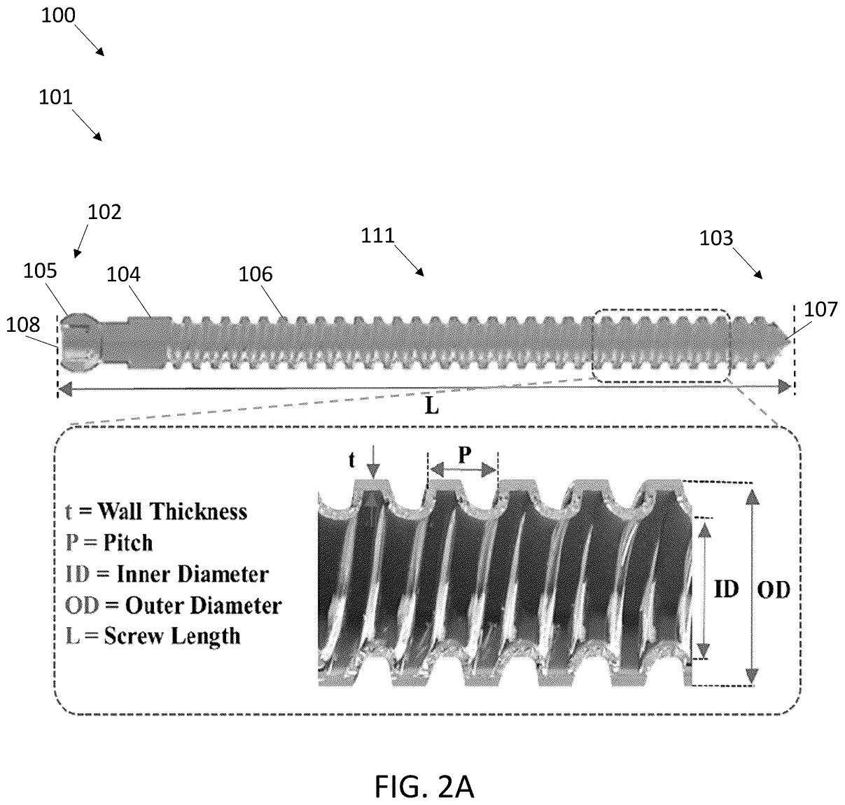

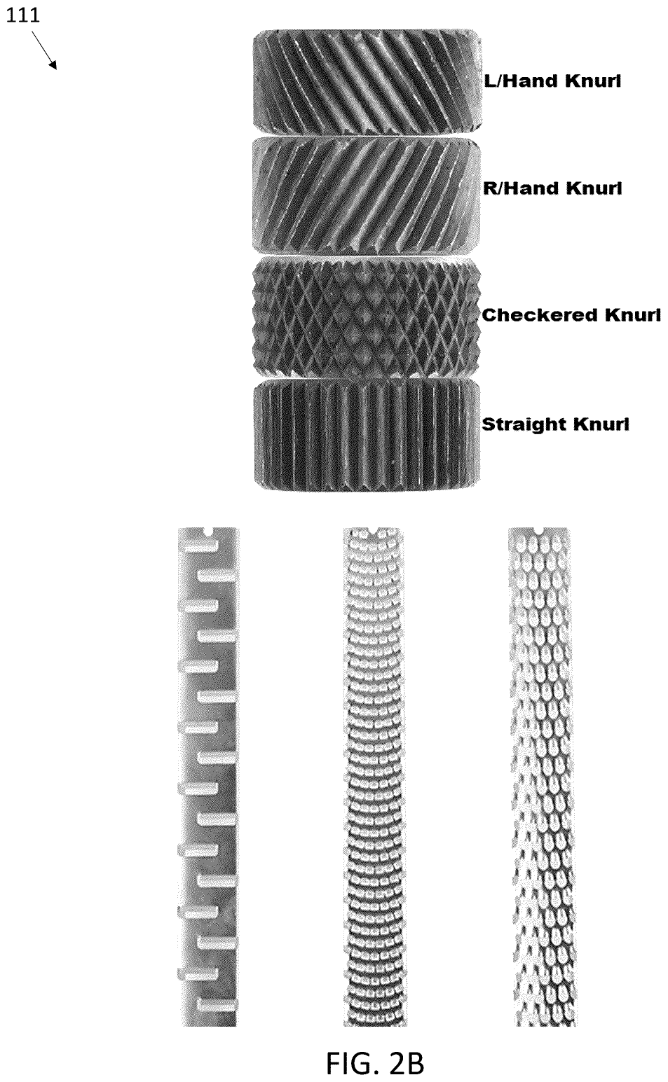

FIG. 1 depicts an exemplary bone fixation system 100, FIG. 2A depicts an exemplary bone fixation device 101 of an exemplary bone fixation system 100, and FIG. 2B depicts exemplary engagement members of the system 100. The system 100 can include a bone fixation device 101, at least one reinforcement wire 110 configured for insertion into the bone fixation device 101, and a castable filler material 109 configured for insertion into the bone fixation device 101. In some embodiments, the system 100 further includes at least one strain gauge configured for insertion into the bone fixation device 101. Furthermore, in some embodiments, additional smart sensors and devices can be inserted into the bone fixation device including, for example, stress sensors, strain sensors, optical fibers, wireless transceivers, and wireless charging devices. These smart sensors and devices can provide live data on forces, stresses, and strains applied to the bone fixation device 100. In some embodiments, the data can be wirelessly transmitted via a wireless transceiver, and power can be supplied via a wireless charging device such as a charging coil.

In some embodiments, the filler material 109 comprises a low melting point alloy such as Field's metal, dental amalgam, and resin-based composites, or any other suitable material or combination thereof, for example. In one embodiment, the at least one reinforcement wire 110 comprises at least one of Nitinol, stainless steel, titanium, carbon fiber, and any other suitable material or combination thereof, for example.

In some exemplary embodiments, the bone fixation device 101 includes a continuum morphable hollow shell structure 104 including a proximal end 102 and a distal end 103, an implantation head 105 at the proximal end 102. In some embodiments, the implantation head 105 includes an aperture 108. In some embodiments, the bone fixation device 101 includes a screw thread 106 positioned external to the shell structure 104 between the implantation head 105 and a screw tip 107 positioned at the distal end 103. The implantation head 105 can be configured as a screw head in some exemplary embodiments. The shell structure 104 can typically be cylindrical, but any suitable shape for implantation into an implantation trajectory can be utilized. In some embodiments, the shell structure 104 is a 3D printed part that is custom made for a specific implantation trajectory. In one exemplary embodiment, the shell structure 104 comprises at least one of ABS, PLA, FormLabs inc. biomaterial (BioMed Clear Resin, BioMed Amber Resin), StrataSys biomaterial (Biocompatible MED625FLX, Biocompatible MED610, Biocompatible VeroGlaze MED620), polyamide, PEEK, titanium, nitinol, cobalt-chrome alloys, and any other suitable material or combination thereof.

For an exemplary cylindrical shell structure 104 embodiment, the shell structure 104 can have an outer diameter in the range of 2 mm to 20 mm, an inner diameter in the range of 1.5 mm to 19.5 mm, and a wall thickness in the range of 0.1 mm to 2 mm, but any suitable values for the outer diameter, inner diameter and wall thickness can be utilized based on the required implantation trajectory and the required implant structural properties needed. The screw thread 106 can have a thread pitch in the range of 1 mm to 2 cm, and the shell structure 104 can have a length in the range of 1 cm to 50 cm, but any suitable thread pitch and shell length can be utilized based on the required implantation trajectory and the required implant structural properties needed. In some embodiments, one or more of the outer diameter, the inner diameter, the wall thickness, the thread pitch, and the thread angle can each vary along the length of the shell structure 104.

While exemplary bone fixation devices 101 and systems 100 of the present invention are described above, the bone fixation devices 101 and systems 100 are nonetheless amenable to any suitable modification to augment their function. For example, in various embodiments, the bone fixation devices 101 and systems 100 can include one or more surface coatings that are configured to enhance pullout strength, biocompatibility, or both. Contemplated coatings include but are not limited to PEEK, PTFE, hydroxyapatite, and the like. In some embodiments, the bone fixation devices and systems can accept a bone cement. In some embodiments, the stiffness of the system 100 is configurable based on a finite element analysis modeled developed based on a target bone mineral density and target bone anatomy by changing the material properties of the castable filler material. In some embodiments, the outer surface of the shell structure 104 includes an engagement member 111 configured to grip onto an implant site, wherein the outer facing surface comprises an engagement structure including but not limited to a screw thread (FIG. 2A), a knurling pattern (FIG. 2B, left), a grating pattern (FIG. 2B, right), and combinations thereof.

In some embodiments, the system 100 can include a plurality of connected bone fixation devices 101 configured to conform to implantation trajectories with complex curvatures. In some embodiments, the plurality of bone fixation devices 101 can be connected in series.

The bone fixation devices 101 and systems 100 of the present invention can be made using any suitable method known in the art. The method of making may vary depending on the materials used. For example, components substantially comprising a metal may be milled from a larger block of metal or may be cast from molten metal. Likewise, components substantially comprising a plastic or polymer may be milled from a larger block, cast, or injection molded. In some embodiments, the components may be made using 3D printing or other additive manufacturing techniques commonly used in the art. In some embodiments, the materials can withstand commonly used sterilization techniques. In some embodiments, the implant devices are constructed from a biocompatible material including but not limited to stainless steel, titanium, nitinol, and combinations and composites thereof.

In some example embodiments, the system 100 can fully exploit a vertebra's curved anatomy by creating a continuum bridge between two bone fragments in a generic shape. This implantation trajectory can help to distribute stresses along the implant to avoid screw loosening and can eliminate the risk of screw pull-out. The screw-like shell structure 104 provides structural flexibility to morph in a generic pre-drilled and threaded trajectory. The hollow shell structure 104 enables shell material between each thread to deform thus allowing the bone fixation device 101 to conform to the shape of an implantation trajectory including straight and/or curved portions. The core filler material 109 and reinforcement wires 110 provide the structural strength and rigidity of the system 100. In some embodiments, the reinforcement wires 110 can be threaded through the aperture 108 of the implantation head 105 to the screw tip 107. Moreover, the internal space of the shell structure 104 can provide sufficient space for embedding sensors and creating a smart bone fixation system 100 toward creating personalized therapeutic and diagnostic capabilities for orthopedic and neurosurgical interventions. A smart bone fixation system 100 can also enable continuous monitoring and wireless transmission of critical intracorporal information toward better understanding of in vivo pathophysiology, bone healing process, implant—tissue interfaces and biomechanics of the implanted morphable implant.

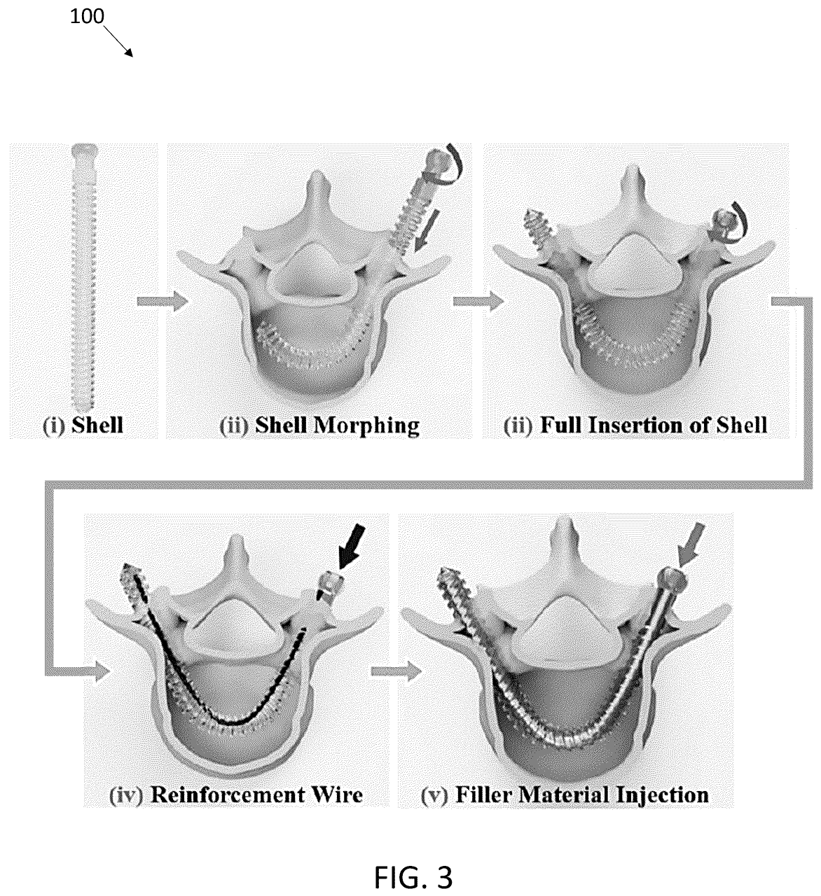

FIG. 3 depicts an exemplary process for implantation and casting of an exemplary reinforced bone fixation system 100. Section (i) shows an exemplary prefabricated bone fixation device 101 is provided. In Section (ii) the exemplary bone fixation device 101 is implanted via a rotational screwing motion into an exemplary pre-drilled threaded U-Shape trajectory. The shell structure 104 of the bone fixation device 101 morphs to follow the implant trajectory. Section (iii) shows the full insertion of the exemplary bone fixation device 101. Section (iv) shows exemplary flexible reinforcement wires 110 being placed inside the shell structure 104. Section (v) shows exemplary filler material 109 being injected inside the shell structure 104 to strengthen and complete the system 100.

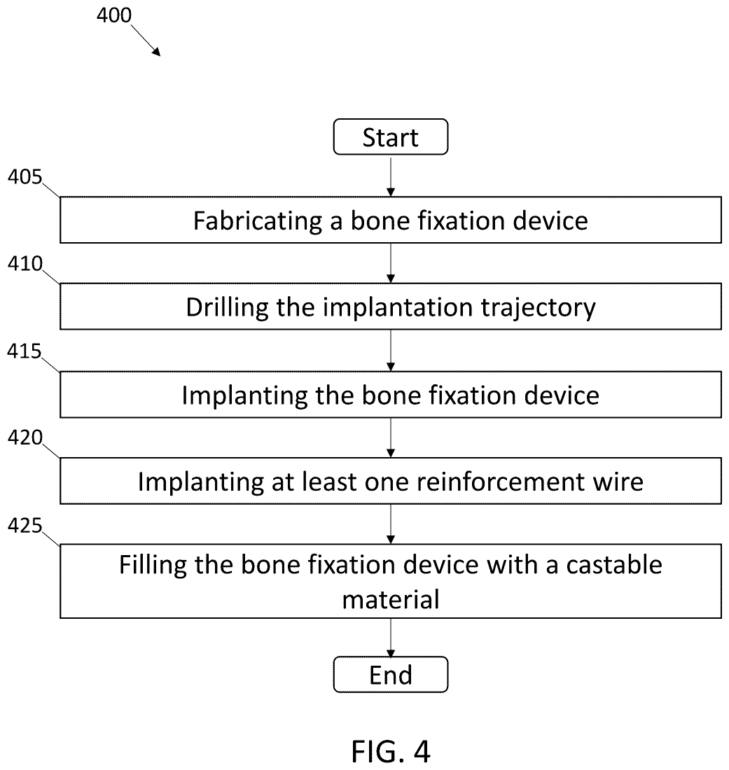

FIG. 4 is a flowchart depicting an exemplary bone fixation method 400. The method 400 begins at Operation 405 where a bone fixation device 101 of a bone fixation system 100 is fabricated. The bone fixation device 101 in some exemplary embodiments can comprise a continuum morphable hollow shell structure 104 including a proximal end 102 and a distal end 103, an implantation head 105 at the proximal end 102 including an aperture 108, and a screw thread 106 positioned external to the shell structure 104 between the implantation head 105 and a screw tip 107 positioned at the distal end 103. The fabrication of the bone fixation device 101 can include a variety of fabrication processes including, but not limited to, casting, molding (such as vacuum molding and injection molding), forming (such as thermoforming, forging, rolling pressing, bending, and extruding), machining (such as milling and turning), joining (such as welding, brazing, soldering and fastening), and additive manufacturing (such as 3D printing), and any other suitable manufacturing processes or combinations thereof, for example.

At Operation 410 the implantation trajectory is drilled. In some embodiments, the drilling is performed with a flexible steerable drilling robot configured to create at least one of a straight, a curved, or a complex trajectory.

At Operation 415 the bone fixation device 101 is implanted into the drilled implantation trajectory. In some embodiments, the device 101 is implanted via a rotational screwing motion by a torque applied to an implantation head 105. In some embodiments the torque is applied directly via a hand, and in other embodiments the torque is applied via a tool interfacing with the implantation head 105 configured as a screw head. In some embodiments, as the implantation is being performed, the bone fixation device 101 is continually morphing along the device's length to follow the drilled implantation trajectory.

At Operation 420 at least one reinforcement wire 110 is implanted within the bone fixation device 101. The at least one wire 110 can be utilized in some exemplary embodiments to provide further structural reinforcement to the system 100. In some embodiments, the wire 110 can be a flexible wire so the wire can follow the implantation trajectory.

The method 400 ends at Operation 425 where the bone fixation device 101 is injected with a castable filler material 109 via an aperture 108. In some embodiments, the filler material 109 comprises a low melting point alloy such as Field's metal, dental amalgam, and resin-based composites, or any other suitable material or combination thereof, for example. In some embodiments, the injecting of the filler material 109 into the bone fixation device 101 is performed in vivo.

In some exemplary embodiments, the method 400 can be optionally preceded by characterizing a target bone tissue of a subject and forming an implantation trajectory based on the characterization. In some embodiments, bone mineral density in a target tissue is measured using quantitative computed tomography (QCT) along with a calibration phantom positioned near a subject. The calibration phantom comprises regions of known Hounsfield units that appear darker with lower densities and lighter with higher densities. Using the calibration phantom, the bone mineral density of the target tissue can be quantified. QCT images are segmented, and a three-dimensional finite element model is constructed based on them such that each element of the model has the material property of the corresponding voxel in the QCT images. In some embodiments, the three-dimensional model can be used to design and analyze a custom implantation path for the bone fixation system 100. Furthermore, it can demarcate osteoporotic regions and low bone mineral density regions of the target tissue for which possible implant trajectories may avoid. While osteoporotic regions may be defined as having a bone mineral density of less than 80 mg/cm 3, it should be understood that any threshold may be used. For example, regions of bone mineral density may be characterized as low relative to the surrounding tissue, such that an optimal implant trajectory favors the higher density tissue over the lower density tissue, even if the lower density tissue has a bone mineral density greater than 80 mg/cm 3. An optimal implant trajectory may avoid osteoporotic regions and low bone mineral density regions, resulting in minimized strain and improved pullout strength when compared to conventional linear screw implantation paths that are unable to evade osteoporotic regions and low bone mineral density regions.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out exemplary embodiments of the present invention, and are not to be construed as limiting in any way the remainder of the disclosure.

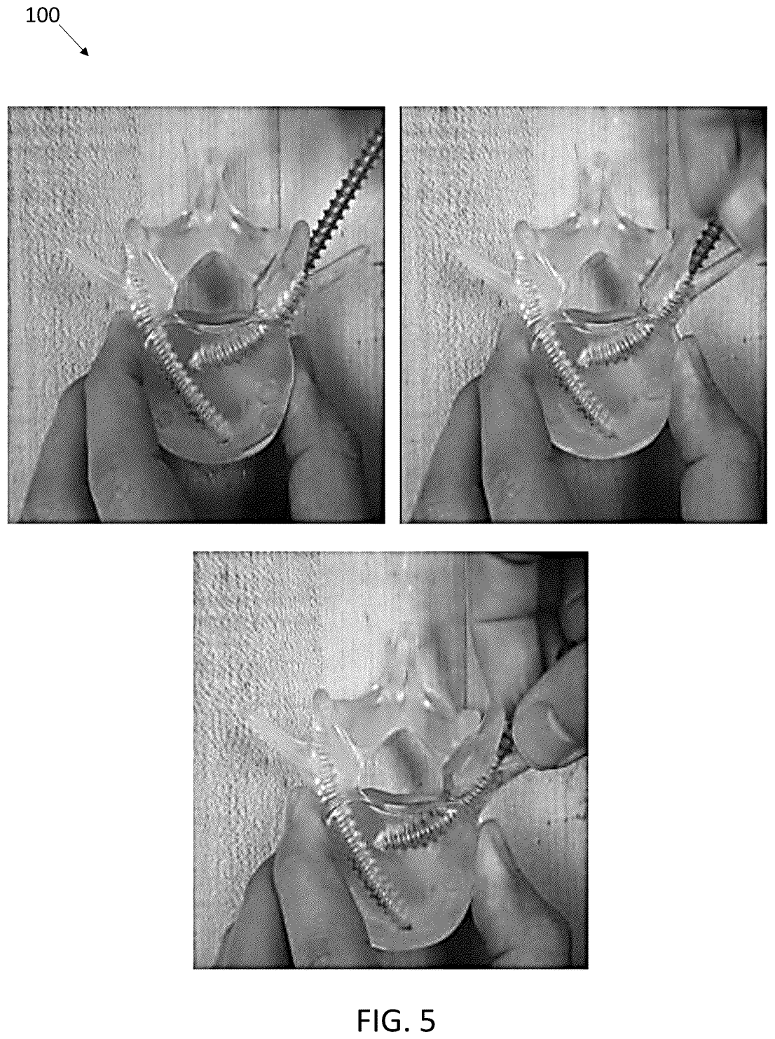

FIGS. 5, 6, 7 and 8 depict exemplary experimental results of exemplary bone fixation systems 100. FIG. 5 shows experimental results of an exemplary bone fixation system configured in a J-shape trajectory implanted in a model L4 vertebra. In the example shown, the bone fixation system 100 is implanted into a J-shaped trajectory. Also, as shown in the figure, a plurality of implantation trajectories can be utilized as necessitated by the implantation site, and thus the bone fixation system 100 can comprise a plurality of bone fixation devices 101.

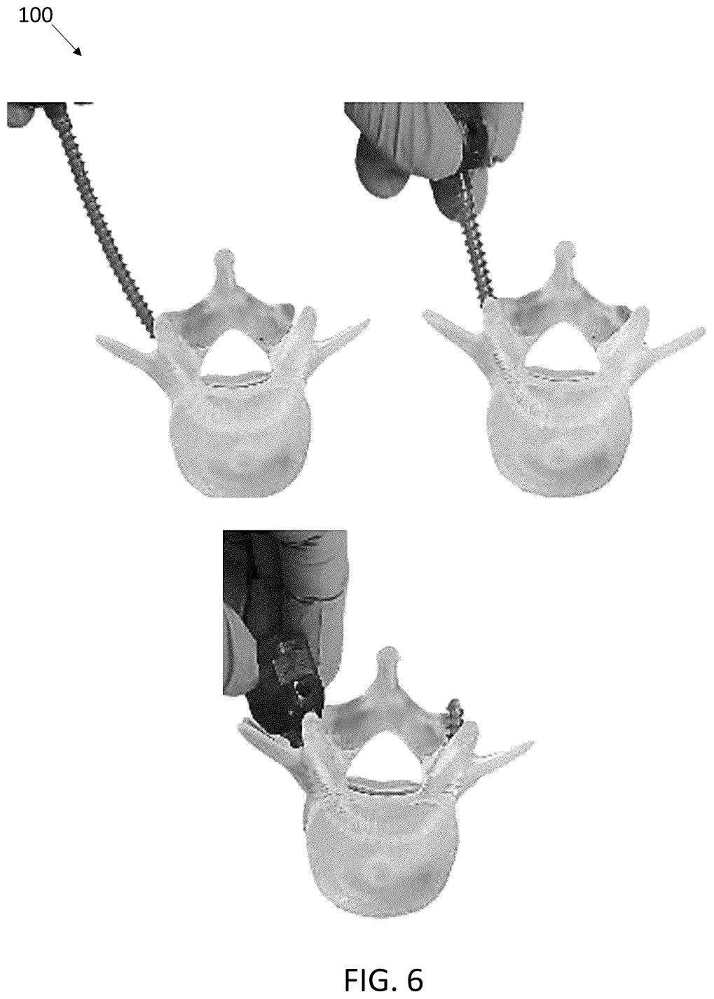

FIG. 6 shows experimental results of an exemplary bone fixation system configured in a U-shaped trajectory implanted in a model L5 vertebra. In the example shown, the bone fixation system 100 is inserted into a U-shaped trajectory with a diameter of 6 mm and a length of 60 mm.

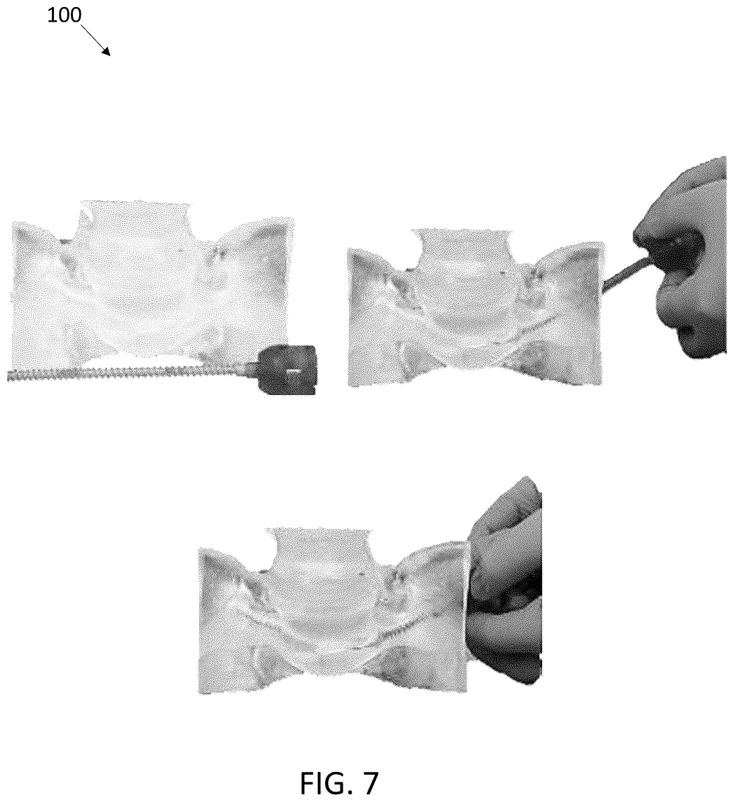

FIG. 7 shows experimental results of an exemplary bone fixation system configured as a long and curved trajectory implanted in a model upper pelvis. In the example shown, the bone fixation device 101 has a length of 150 mm.

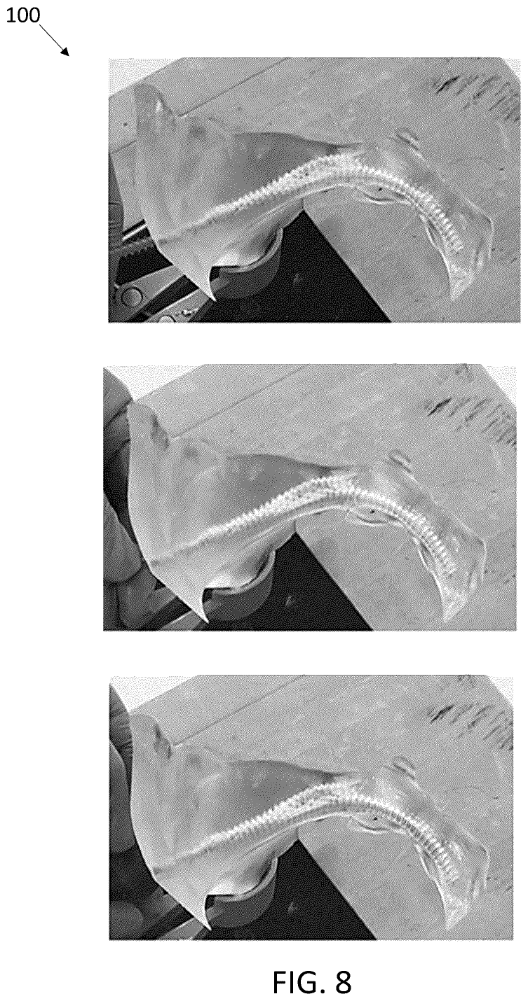

FIG. 8 shows experimental results of an exemplary bone fixation system configured as curved and long trajectory implanted in a model right pelvis. In the example shown, the bone fixation device 101 has a length of 230 mm.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A bone fixation device, comprising:
a continuum morphable hollow shell structure including a proximal end and a distal end;
an implantation head at the proximal end including an aperture; and
a screw thread positioned external to the shell structure between the implantation head and a screw tip positioned at the distal end.

2. The device of claim 1, wherein the implantation head comprises a screw head.

3. The device of claim 1, wherein the shell structure is cylindrical.

4. The device of claim 1, wherein the shell structure has an outer diameter in the range of 2 mm to 20 mm, an inner diameter in the range of 1.5 mm to 19.5 mm, and a wall thickness in the range of 0.1 mm to 2 mm.

5. The device of claim 1, wherein the screw thread has a thread pitch in the range of 1 mm to 2 cm.

6. The device of claim 1, wherein the shell structure has a length in the range of 1 cm to 50 cm.

7. The device of claim 1, wherein the shell structure comprises at least one of ABS, PLA, biomaterial, polyamide, PEEK, titanium, nitinol, and cobalt-chrome alloys.

8. The device of claim 1, wherein the shell structure is a 3D printed part.

9. A bone fixation system, comprising:
a bone fixation device including a continuum morphable hollow shell structure including a proximal end and a distal end, an implantation head at the proximal end including an aperture, and a screw thread positioned external to the shell structure between the implantation head and a screw tip positioned at the distal end;
at least one reinforcement wire configured for insertion into the bone fixation device; and
a castable filler material configured for insertion into the bone fixation device.

10. The system of claim 9, further comprising at least one strain gauge configured for insertion into the bone fixation device.

11. The system of claim 9, wherein the filler material comprises a low melting point alloy.

12. The system of claim 9, wherein the filler material comprises at least one of Field's metal, dental amalgam, and resin-based composite.

13. The system of claim 9, wherein the at least one reinforcement wire comprises at least one of Nitinol, stainless steel, titanium, and carbon fiber.

14. The system of claim 9, wherein the stiffness of the system is configurable based on a finite element analysis modeled developed based on a target bone mineral density and target bone anatomy by changing the material properties of the castable filler material.

15. A bone fixation method, comprising:
fabricating a bone fixation device including a continuum morphable hollow shell structure including a proximal end and a distal end, an implantation head at the proximal end including an aperture, and a screw thread positioned external to the shell structure between the implantation head and a screw tip positioned at the distal end;
drilling an implantation trajectory;
implanting the bone fixation device;
implanting at least one reinforcement wire within the bone fixation device; and
injecting the bone fixation device with a castable filler material via an aperture of the bone fixation device.

16. The method of claim 15, further comprising characterizing a target bone tissue including identifying regions of osteoporotic bone and bone with low mineral density, and forming the implantation trajectory based on the characterization.

17. The method of claim 16, wherein the implantation trajectory is configured to avoid the identified regions of osteoporotic bone and bone with low mineral density.

18. The method of claim 16, wherein the step of characterizing the target bone tissue comprises the steps of:
performing one or more quantitative computed tomography (QCT) scans on the target bone tissue;
converting the one or more QCT scans into a three-dimensional finite element model of the target bone tissue; and
demarcating osteoporotic regions or low bone mineral density regions in the three-dimensional finite element model.

19. The method of claim 15, wherein the bone fixation device is fabricated by at least one of additive manufacturing and molding.

20. The method of claim 15, wherein the filler material comprises a low melting point alloy.

21. The method of claim 15, wherein the filler material comprises at least one of Field's metal, dental amalgam, and resin-based composite.

22. The method of claim 15, wherein the injecting of the filler material into the bone fixation device is performed in vivo.

23. The method of claim 15, wherein the at least one reinforcement wire comprises at least one of Nitinol, stainless steel, titanium, and carbon fiber.

24. The method of claim 15, wherein the drilling is performed with a flexible steerable drilling robot configured to create at least one of a straight, a curved, or a complex trajectory.
